(19) United States
(12) Patent Application Publication
(10) Pub. No.: US 2019/0370424 A1
(43) Pub. Date: Dec. 5, 2019

(54) DETERMINING MECHANICAL PROPERTIES OF COMPOSITE MATERIALS

(71) Applicant: Saudi Arabian Oil Company, Dhahran (SA)

(72) Inventors: Yanhui Han, Katy, TX (US); Younane N. Abousleiman, Norman, OK (US)

(21) Appl. No.: 15/994,734

(22) Filed: May 31, 2018

(51) Int. Cl. G06F 17/50; G06F 17/12; G01N 33/24; G01N 3/00; G06T 17/10

(52) U.S. Cl. CPC G06F 17/5018; G06F 17/12; G01N 2203/0075; G01N 3/00; G06T 17/10; G01N 33/24

(57) ABSTRACT

A real composite material includes multiple real materials interspersed to form the real composite material. Multiple mechanical properties of the respective real materials are determined by one or more real mechanical tests. The real composite material is scanned to form a digitized model of the real composite material. The digitized model of the real composite material includes digitized models of the real materials. The mechanical properties (determined by the one or more real mechanical tests) are assigned to the corresponding digitized models of the respective real materials, resulting in a modified digitized model of the real composite material. One or more simulated mechanical tests are performed on the modified digitized model of the real composite material. A mechanical property of the real composite material is determined based on results of the one or more simulated mechanical tests. The mechanical property of the real composite material is provided.

### TECHNICAL FIELD

This disclosure relates to determining properties of composite materials.

### BACKGROUND

A composite material is a material made from two or more constituent materials whose physical or chemical properties are different. Composite materials can be artificial, such as reinforced concrete, which is typically made of wire mesh, steel bars, cement, gravel, and sand. Composite materials can be natural, such as kerogen rich shales, which consist of clay particles, quartz grains, and organic matter. Mechanical properties of various materials are often important parameters in research, design, and analyses in the science and engineering industries.

### SUMMARY

This disclosure describes technologies relating to determining mechanical properties of composite materials. Certain aspects of the subject matter described here can be implemented as a method. A real composite material includes multiple real materials interspersed to form the real composite material. Multiple mechanical properties of the respective real materials are determined by one or more real mechanical tests. The real composite material is scanned to form a digitized model of the real composite material. The digitized model of the real composite material includes digitized models of the real materials. The mechanical properties (determined by the one or more real mechanical tests) are assigned to the corresponding digitized models of the respective real materials, resulting in a modified digitized model of the real composite material. One or more simulated mechanical tests are performed on the modified digitized model of the real composite material. A mechanical property of the real composite material is determined based on results of the one or more simulated mechanical tests. The mechanical property of the real composite material is provided.

This, and other aspects, can include one or more of the following features.

Scanning the real composite material can include scanning electron microscopy (SEM) imaging or micro computed tomography (micro-CT) imaging to create multiple images. The digitized model of the real composite material can be formed using the images.

The digitized model of the real composite material can be formed by identifying, in the images, digitized portions that correspond to each of the real materials.

Assigning the determined mechanical properties to the corresponding digitized models of the respective real materials can include assigning the determined mechanical properties to the digitized portions identified as corresponding to each of the real materials.

The real composite material can include a core plug obtained from a subterranean zone.

The real materials can include organic material.

Certain aspects of the subject matter described here can be implemented as a method. A composite material includes multiple constituents interspersed to form the composite material. A digitized model of the composite material is formed. The digitized model includes multiple digitized sub-models corresponding to the respective constituents. One or more mechanical properties are assigned to each of the digitized sub-models to form a modified digitized model of the composite material. The assigned mechanical properties each correspond to mechanical properties of their respective constituents. A simulated mechanical test is performed on the modified digitized model to determine a mechanical property of the composite material. The mechanical property of the composite material is provided.

This, and other aspects, can include one or more of the following features. Forming the digitized model of the composite material can include scanning the composite material to obtain multiple images. The images can be compiled to form the digitized model of the composite material.

Scanning the composite material can include SEM imaging or micro-CT imaging.

The composite material can include a core plug obtained from a subterranean zone.

The constituents can include organic material.

One or more mechanical test can be performed to determine one or more mechanical properties of each of the constituents. The one or more mechanical properties determined by performing the one or more mechanical tests can be the one or more mechanical properties assigned to each of the digitized sub-models.

Certain aspects of the subject matter described here can be implemented as a method. A composite material includes multiple constituents interspersed to form the composite material. Mechanical properties of each of the constituents are determined. The composite material is scanned to obtain at least one image. The at least one image is processed to form a digitized model of the composite material. The digitized model includes multiple digitized sub-models corresponding to the respective constituents. The digitized model is modified to include the determined mechanical properties of each of the constituents, resulting in a modified digitized model. A mechanical property of the composite material is determined based on results of a simulated mechanical test on the modified digitized model. The mechanical property of the composite material is provided.

This, and other aspects, can include one or more of the following features. The composite material can include a core plug obtained from a subterranean zone.

The constituents can include organic material.

The details of one or more implementations of the subject matter of this disclosure are set forth in the accompanying drawings and the description. Other features, aspects, and advantages of the subject matter will become apparent from the description, the drawings, and the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A, 1B, and 1C are schematic diagrams of examples of composite materials.

FIG. 2 is a flow chart of a method for determining properties of composite materials.

FIG. 3 is an example of a model of a composite material.

FIGS. 4A and 4B are graphs showing results of a simulated mechanical test.

FIG. 5 is a graph showing results of results of simulated mechanical tests.

FIG. 6 is a block diagram of an example computer system.

### DETAILED DESCRIPTION

In scientific research and engineering activities where composite materials are involved, it can be important to know the mechanical properties of such composite materials. For example, to calculate the deflection and loading capacity of a concrete structural beam or pillar of a building, the stiffness and strength properties of the reinforced concrete used to make these structures can be useful. As another example, to safely drill through shales and efficiently stimulate production from unconventional reservoirs, knowing mechanical properties and behaviors of kerogen-rich shales can be useful.

This disclosure describes determining mechanical properties of real composite materials using digitized models of the real composite materials. In some implementations, real composite materials can be scanned to produce digitized models. The real composite materials are made up of constituent materials, each of which has respective mechanical properties. Correspondingly, the digitized model is also made up of digitized models of the constituent materials. Portions of the digitized model that correspond to the constituent materials are assigned mechanical properties of those materials. In this manner, various parameters corresponding to characteristics of the composite material can be inputted into the digitized model. The resulting digitized model can emulate the behavior of the composite material in response to various actions, such as applied forces or stresses. In this disclosure, the term “real” refers to materials or actions that exist in the real world (as opposed to a digital space). Thus, a real material is a physical, tangible material with a physical, tangible structure. An example of a real material is a core plug obtained from a subterranean formation. The core plug obtained from a subterranean formation can also be a composite material. For example, if the core plug was obtained from a sandstone reservoir, the core plug can include various materials, such as quartz, feldspar, and other rock fragments or minerals. A real action or real experiment is a physical, tangible action performed on a real material. An example of a real action is an indentation test performed on a real material, for example, the core plug obtained from the subterranean formation.

Methods for creating a digitized model of a composite material are described. Mechanical properties of the composite material can be determined by running simulation tests on the digitized model. Various tests on nano- and micro-scales (that is, on the order of nanometers and micrometers) are run to measure mechanical properties of individual phases or components present in the composite material, and the data obtained is post-processed and mapped to corresponding components in the digitized model. A sample of the composite material can be scanned, for example, using focus ion beam scanning electron microscopy (FIB-SEM) or X-ray computerized tomography (CT), to construct a three-dimensional image of the sample. Various simulation tests (for example, triaxial compression test, direct shear test, direct tensile test, Brazilian test, and flexural test) can then be run on the digitized model to determine the mechanical properties of the composite material, such as Young's modulus, Poisson's ratio, tensile strength, cohesive strength, toughness, angle of internal friction, and dilation angle.

The subject matter described in this disclosure can be implemented in particular implementations, so as to realize one or more of the following advantages. Because a digitized model of the composite material is formed, multiple simulated tests can be performed on the digitized model to determine characteristics and behavior of the composite material. A simulated test can be repeated multiple times with different sets of parameters to determine a sensitivity of a mechanical property to a parameter. Results of a simulated test performed on the digitized model can be compared to results of a real mechanical test performed on the composite material. The comparison of results can be used to optimize the digitized model. Multiple, different simulated tests can be performed on the digitized model without concern of damaging the digitized model. In comparison, some mechanical tests destroy or otherwise alter the material being tested, meaning that additional tests cannot be performed to produce results that accurately portray the characteristics and behavior of the material. In contrast to real laboratory tests, simulated tests are not restricted by sample size. For example, a simulated tri-axial test can be performed on nano-scale digitized model of a rock sample, whereas performing a real tri-axial test on a nano-scale rock sample can be prohibitively difficult to perform. Additionally, once a digitized model is obtained, the simulated tests can be performed at lower costs and more quickly in comparison to real tests.

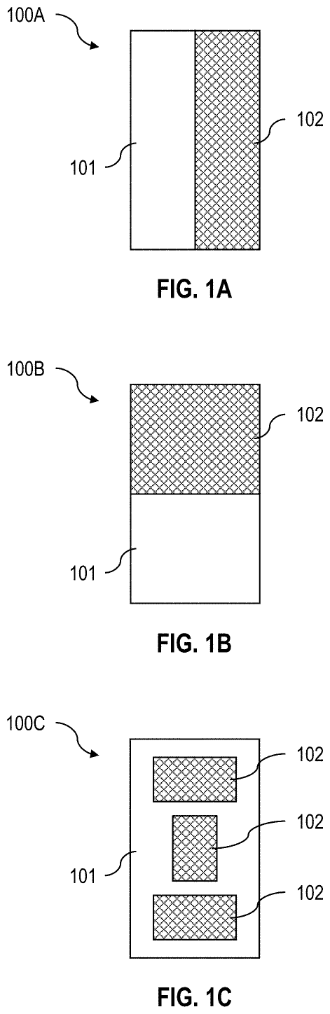

Characteristics, such as mechanical properties, of a composite material can be significantly different from those of its constituents. Generally, mechanical properties and behaviors of composite materials depend on various factors, such as the quantity and distribution of its constituent materials. FIGS. 1A, 1B, and 1C show various examples of composite materials (100A, 100B, and 100C, respectively). The composite materials (100A, 100B, 100C) are made up of at least two constituent materials, for example, a first constituent 101 and a second constituent 102. A non-limiting example of a composite material is a rock that includes organic material, such as a kerogen-rich shale. For example, for the composite material 100A, the first constituent 101 can be quartz, and the second constituent 102 can be organic material, such as kerogen. As shown in FIGS. 1A, 1B, and 1C, the composite materials (100A, 100B, 100C) can have different amounts and distributions of constituents (101, 102). The composite materials 100A, 100B, and 100C shown in FIGS. 1A, 1B, and 1C are simplified schematic examples, and the distribution and concentrations of constituents in real composite materials can be different from these examples. Referring back to the example of kerogen-rich shale, a real sample of kerogen-rich shale will likely comprise a shale rock sample with organic material (such as kerogen), clay particles, quartz, calcite, and other materials, all interspersed among each other (in contrast with having substantial regions containing only one constituent, as is schematically shown in FIGS. 1A, 1B, and 1C).

One technique to determine mechanical properties of a composite material is to perform real experiments on real samples of the material. For example, a uniaxial compression test can be performed on the composite materials, for example, the composite materials 100A, 100B, and 100C. The uniaxial compression test includes applying a force on the composite material (100A, 100B, or 100C) along a single axis of the composite material (for example, a compression force along a longitudinal axis of the composite material). The Young's modulus, also referred as the elastic modulus, is a measure of stiffness (that is, rigidity or ability to resist deformation in response to an applied force) of a solid material. The Young's modulus can be determined based on the results of a uniaxial compression test. For example, the Young's modulus of a composite material (for example, 100A) can be calculated by Equation 1:

$$E = \frac{FL}{A Δ L} \qquad ( 1 )$$

where F is the force applied in the uniaxial compression test, L is the initial vertical length of the composite material, A is the cross-sectional area of the composite material that is perpendicular to the applied force F, and Δl, is the vertical deformation (or displacement) of the surface upon which the force F is applied. Poisson's ratio is a measure of the Poisson effect, a phenomenon in which a material tends to expand in directions perpendicular to the direction of compression. Poisson's ratio is the negative value of the ratio of transverse strain to axial strain.

The first constituent 101 can have, for example, a Young's modulus of 50 gigapascals (GPa) and a Poisson's ratio of 0.23. The second constituent 102 can have, for example, a Young's modulus of 5 GPa and a Poisson's ratio of 0.25. Even though the composite materials 100A, 100B, and 100C are made up of the same constituents 101 and 102, the composite materials 100A, 100B, and 100C can have different mechanical properties and behaviors due to the differences in distribution of the constituents 101 and 102. For example, assuming that the sizes of the composite materials (100A, 100B, 100C) are the same, identical uniaxial compression tests can be performed on each of the composite materials 100A, 100B, and 100C (that is, uniaxial compression tests with the same applied force), and the Young's moduli calculated from the results of the tests can be 27.5 GPa, 9.1 GPa, and 18.6 GPa for the composite materials 100A, 100B, and 100C, respectively.

Another technique to determine mechanical properties of a composite material is to perform simulated mechanical tests on a digitized model of the composite material. Digitized models can include parameters that correspond to known mechanical properties of the composite material (such as mechanical properties of the individual constituents that make up the composite material), so that the digitized model can mimic the behavior of the composite material in response to external stimuli, such as mechanical shear. The inclusion of such parameters into such digitized models form enhanced representations of the composite material. A simulated mechanical test can include, for example, a simulated compressive force on the digitized model. The behavior of the digitized model in response to the simulated compressive force can be observed. The behavior of the digitized model during the simulated test can be expected to match the behavior of the composite material undergoing a similar stimulus (for example, an actual compressive force). Because the digitized model is expected to emulate the behavior of the composite material, properties of the composite material can be inferred from results of the simulated test.

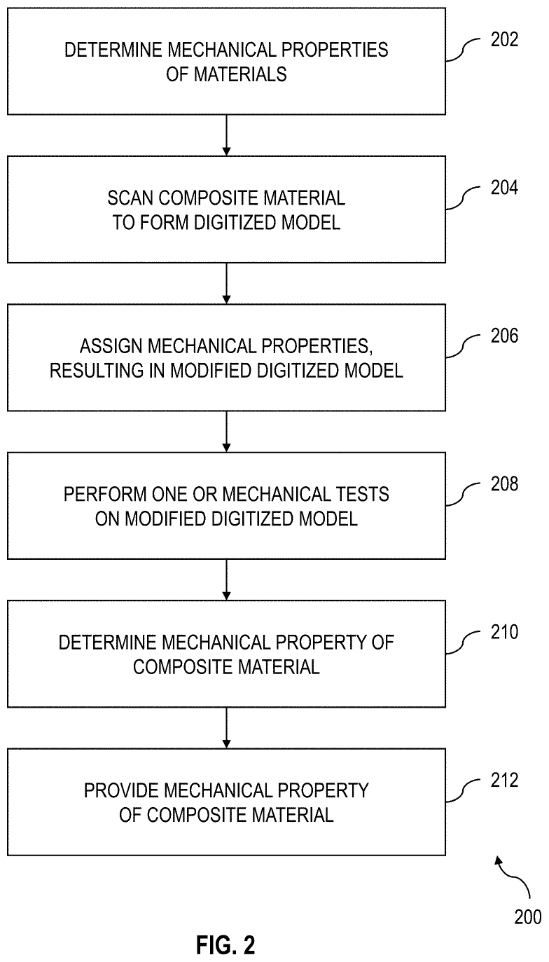

FIG. 2 shows a flow chart of a method 200 for determining mechanical properties of a real composite material (for example, composite material 100A). The real composite material includes at least two real materials (that is, constituents) interspersed to form the real composite material. The core plug can be prepared, cleaned, and shaped to be suitable for the equipment being used to carry out the method 200.

At 202, mechanical properties of the various real materials that make up the real composite material are determined by one or more real mechanical tests. The one or more real mechanical tests can include, for example, a single stage indentation test, a multistage indentation test, a micro-cantilever beam test, a micro-scratch test, micro-pillar compression test, direct tension test, direct shear test, or any combination of these. The one or more real mechanical tests can be performed to determine mechanical properties, such as Young's modulus, tensile strength, and shear strength parameters, such as cohesive strength, angle of internal friction, and dilation angle.

The mechanical properties of each of the real materials that make up the real composite material can be determined. For example, a multi-stage nano-indentation test can be performed on a real composite material, such as a core plug. In a typical nano-indentation test, if the penetration (that is, deformation) is well-confined by a single constituent (for example, kerogen) of the composite material (such as the core plug), then the loading-unloading force-displacement curves obtained from the indentation test can be inferred to convey the mechanical response of that constituent. Therefore, the mechanical properties (such as the stiffness and other strength properties) of the indented constituent can be extracted from the recorded curves by using analytical or numerical models. Determining mechanical properties of the individual constituents making up the core plug can include comparing results of the multi-stage nano-indentation test with a numerical model. The numerical model can include parameters corresponding to mechanical properties of the individual constituents. These parameters can be adjusted until the results of the multi-stage nano-indentation test match with the numerical model. The adjusted parameters (that allow the numerical model to match the results of the multi-stage nano-indentation test) correspond to the actual mechanical properties of the individual constituents. Thus, the adjusted parameters can be used to determine the mechanical properties of the various real materials that make up the real composite material. Further details of such single-stage and multi-stage indentation tests that can be performed to determine mechanical properties can be found in U.S. Patent Publication 2017/0370895.

At 204, the real composite material is scanned to form a digitized model of the real composite material. In some implementations, the real composite material is scanned at 204 before performing the one or more real mechanical tests to determine the mechanical properties of the various real materials making up the real composite material at 202. The digitized model of the real composite material includes digitized models of the real materials making up the real composite material. For example, the digitized model of the real composite material can include various, smaller digitized models (that is, digitized portions or sub-models) of the real materials located and distributed in the digitized model of the real composite material in the same locations and distributions of the real materials in the real composite material. Referring back to FIG. 1A, for composite material 100A, the digitized model can include a digitized model of the first constituent 101 and a digitized model of the second constituent 102. As described later, the locations and distributions of the digitized model of the first constituent 101 and the digitized model of the second constituent 102 in the digitized model of the composite material 100A can correlate to the locations and distributions of the first constituent 101 and the second constituent 102 in the composite material 100A.

In some implementations, the real composite material is scanned by electron microscopy (SEM) imaging (such as SEM mosaic imaging), micro computed tomography (micro-CT) imaging (such as HeliScan™ micro-CT), mineralogy mapping, or a combination of these. The imaging techniques can be used to create one or more images of the real composite material. The digitized model can then be formed using the images obtained by scanning the real composite material. The images can be compiled to form the digitized model. In some implementations, multiple cross-sectional images are taken of the real composite material, and the cross-sectional images are compiled to form the digitized model. In some implementations, the images are processed and compiled to form the digitized model. The digitized model can be constructed by generating a three-dimensional image using scanning equipment (such as micro-CT), image segmentation (for example, using Avizo®), and digital rock buildup using various mapping methods. The images obtained by scanning the real composite material can be processed to identify regions (that is, digitized portions) that correspond to the each of the respective real materials making up the real composite material. For example, raw image data can be exported to Avizo® Fire, and a suitable filter (for example, non-local means filter) can be applied. The various constituents can then be identified by using various image segmentation algorithms, such as multilevel thresholding. The resolution (that is, the pixel size) of the digitized model can range between approximately 1 nanometer (nm) to approximately 100 micrometers (μm). For example, the resolution of the digitized model is 10 nm, 100 nm, 1 or 10 μm. In this disclosure, “approximately” means a deviation or allowance of up to 10 percent (%) and any variation from a mentioned value is within the tolerance limits of any machinery used to manufacture the part.

At 206, the mechanical properties determined at 202 are assigned to the corresponding digitized models of the respective real materials, resulting in a modified digitized model of the real composite material. The mechanical properties determined at 204 can be assigned to the digitized portions identified as corresponding to each of the respective real materials. For example, mechanical properties such as Young's modulus and Poisson's ratio of kerogen (an example of a real material) are assigned to the digitized portions which correspond to the regions of a kerogen-rich shale (an example of a real composite material) that include kerogen. Referring back to FIG. 1A, a digitized model of the composite material 100A can include mechanical properties of the first constituent 101 and mechanical properties of the second constituent 102. The mechanical properties of the first constituent 101 can be assigned to a portion of the digitized model (for example, a digitized sub-model) that corresponds to the distribution and location of the first constituent 101 in the composite material 100A. The mechanical properties of the second constituent 102 can be assigned to portions of the digitized model (for example, one or more digitized sub-models) that correspond to the distribution and locations of the second constituent 102 in the composite material 100A. For example, the digitized model can be made of voxels that can be stored in a linked list format. The voxels of the digitized model that correspond to same constituent can be assigned the same group name. When assigning mechanical properties to the digitized model, each of the voxels making up the digitized model are run through, and voxels that are assigned the same group name are assigned with the same mechanical properties.

Referring back to FIG. 2 at 208, one or more simulated mechanical tests are performed on the modified digitized model of the real composite material. The simulated tests can be performed, for example, using a computational geomechanics software, such as FLAC3D™ developed and distributed by Itasca Consulting Group, Inc. A useful feature of FLAC3D™ is that the software includes a built-in programming language called FISH (also referred as FLAC-ISH). The simulated tests can be implemented in FISH. FISH can be used as a high-level programming language (similar to Fortran or C++), and because FISH is available as an embedded programming language in FLAC3D™, FISH can be used to access and modify data and computational procedures of the digitized models developed with FLAC3D™. The simulated tests can include generating a mesh, assignment materials (such as assigning constituents and mechanical properties of the individual constituents), initiating the digitized model, enforcing boundary conditions, monitoring scheme installation, and post-processing the results of the simulated tests. The one or more simulated mechanical tests can include, for example, a uniaxial compression test, a triaxial compression test, a direct shear test, a direct tension test, a Brazilian test, and an iso-compression test. Because the mechanical properties of the various constituents were assigned to the digitized model at 206, the modified digitized model is an enhanced representation of the real composite material. The modified digitized model can emulate the behavior of the real composite material in response to simulated external stimuli, such as an applied force or stress.

At 210, a mechanical property of the real composite material is determined based on results of the one or more simulated mechanical tests performed at 208. For example, the compressive strength, the angle of internal friction, and the cohesion of the real composite material can be determined based on results of a triaxial compression test performed on the digitized model. Similarly, tensile strength can be determined based on results of a direct tension test or a Brazilian test. It is noted that many constitutive models have been proposed for geological materials, with the Mohr-Coulomb model being one of the most widely used for elastoplastic modeling of geological materials. In some implementations, other models, such as Heok-Brown, modified Lade, Drucker-Pager, Strain-Softening/Hardening, Cam-Clay, modified Cam-Clay, Ubiquitous Joint, can be used to model the composite material. Because the mechanical properties of the various constituents were assigned to the digitized model at 206, the results of the one or more simulated mechanical tests performed at 208 can be reasonably expected to match the results of a real mechanical test performed on the real composite material. One or more mechanical properties of the real composite material can be determined at 210.

In some implementations, additional mechanical properties of the various constituent materials making up the composite material can be determined from the results of the one or more simulated mechanical tests performed at 208. For example, the Young's moduli of each of the constituents can be inputted into the digitized model to form a modified digitized model, and a different property, such as the bulk modulus, of each of the constituents can be determined from the results of a simulated mechanical test on the modified digitized model.

At 212, the mechanical property determined at 210 is provided, for example, as an output, such as digital data, visual information, audio information, or a combination of information. For example, the mechanical property can be provided as an output by a computer (described in more detail later). The mechanical properties determined at 210 can be used, for example, to develop a strategy for drilling a well or completing a well. The mechanical properties determined at 210 can, for example, reveal the level of compaction of a subterranean zone or be used to assess the integrity of a well. The mechanical properties determined at 210 can be used in engineering design. For example, both compressive strength and tensile strength can be useful inputs for drilling and sand control design. As another example, tensile strength can be used in hydraulic fracturing simulation. As another example, Young's modulus can be used to predict reservoir compaction and surface subsidence.

### Example 1

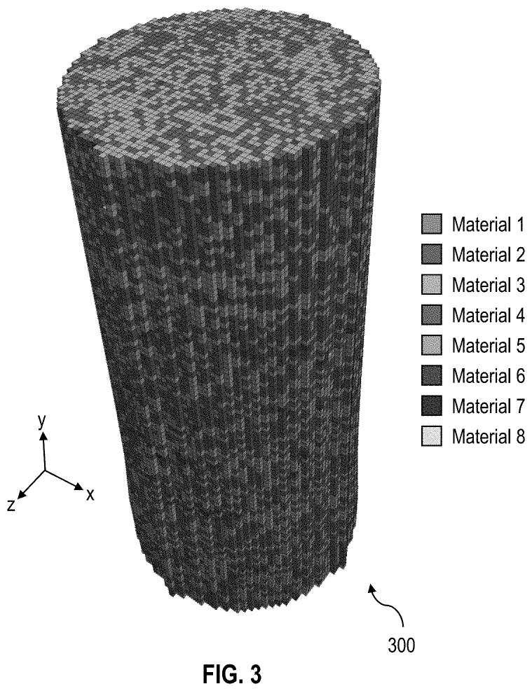

FIG. 3 is an example of a digitized model 300 of a composite material. In the example shown in FIG. 3, the digitized model 300 includes eight different constituent materials, each represented by a different color. The digitized model 300 was constructed by micro-CT scanning a real rock sample (Boise sandstone), which was cylindrical in shape and had a length of 14.92 centimeters (cm) and a diameter of 7.46 cm. The digitized model 300 was constructed using Avizo® Fire. The digitized model 300 had 100 elements (that is, voxels) in the y-direction (corresponding to the length of the real rock sample) and a maximum of 50 elements in the x- and z-directions (corresponding to the diameter of the real rock sample). Therefore, each element (voxel) had representative dimensions of 1.492 millimeters (mm) by 1.492 mm by 1.492 mm. The digitized model 300 included multiple digitized sub-models corresponding to the respective constituent materials that made up the composite material (the real rock sample of Boise sandstone).

The digitized model 300 included the various mechanical properties of the constituents making up the composite material. The mechanical properties were assigned to the various digitized sub-models, which correspond to the respective constituents. The mechanical properties of the constituent materials were measured at a micro-scale (that is, at a scale on the order of micrometers) and included in the digitized model 300. The mechanical properties of the constituent materials were determined by performing multi-stage indentation tests and numerical modeling as described in U.S. Patent Publication 2017/0370895. The properties are provided in the following table.

| Component | B | V | ρ | κ | λ | f_{c} | P₀ | P_{c} | V₀ | ν |
|---|---|---|---|---|---|---|---|---|---|---|
| 1 | 40 | 1.26 | 2.31 | 0.0054 | 0.167 | 0.929 | 22.066 | 166.2 | 1.575 | 0.16 |
| 2 | 40 | 1.32 | 2.26 | 0.0065 | 0.167 | 0.929 | 22.066 | 125.7 | 1.575 | 0.16 |
| 3 | 40 | 1.37 | 2.21 | 0.0076 | 0.167 | 0.929 | 22.066 | 92.6 | 1.575 | 0.16 |
| 4 | 40 | 1.44 | 2.15 | 0.0088 | 0.167 | 0.929 | 22.066 | 66.1 | 1.575 | 0.16 |
| 5 | 40 | 1.48 | 2.13 | 0.0101 | 0.167 | 0.929 | 22.066 | 45.6 | 1.575 | 0.16 |
| 6 | 40 | 1.55 | 2.05 | 0.0116 | 0.167 | 0.929 | 22.066 | 30.1 | 1.575 | 0.16 |
| 7 | 40 | 1.63 | 2.02 | 0.0135 | 0.167 | 0.929 | 22.066 | 18.9 | 1.575 | 0.16 |
| 8 | 40 | 1.73 | 1.97 | 0.0151 | 0.167 | 0.929 | 22.066 | 11.2 | 1.575 | 0.16 |

B is maximum elastic bulk modulus (a measure of resistance to compressibility).

V is initial specific volume (a ratio of bulk volume to volume of solid particles of component).

ρ is mass density in grams per cubic centimeter.

κ is the slope of elastic swelling (mechanical property of a Cam-Clay type material indicating the compressibility of an over-consolidated clay-type material).

λ is the slope of the normal consolidation line (mechanical property of a Cam-Clay type material indicating the compressibility of a normally consolidated clay-type material).

f_{c} is a frictional constant (a measure of compressive shearing strength).

P₀ is the reference pressure in megapascals (MPa).

P_{c} is the pre-consolidation pressure in MPa.

V₀ is the specific volume at reference pressure P₀.

v is Poisson's ratio.

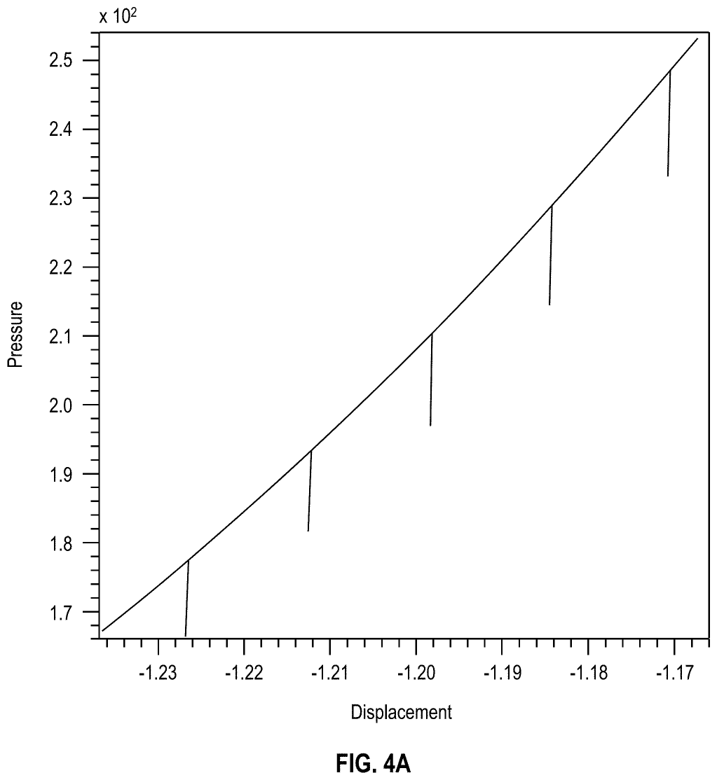

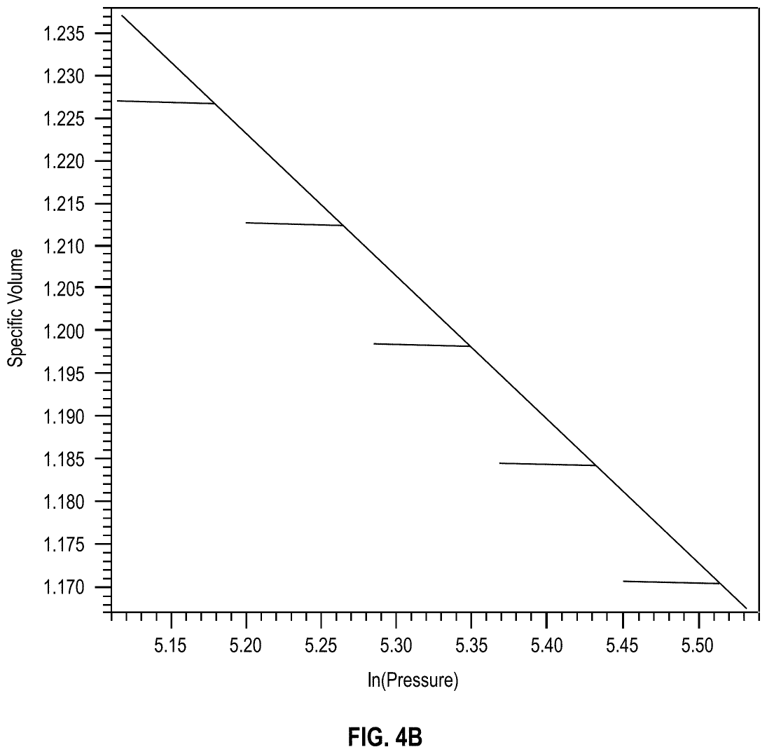

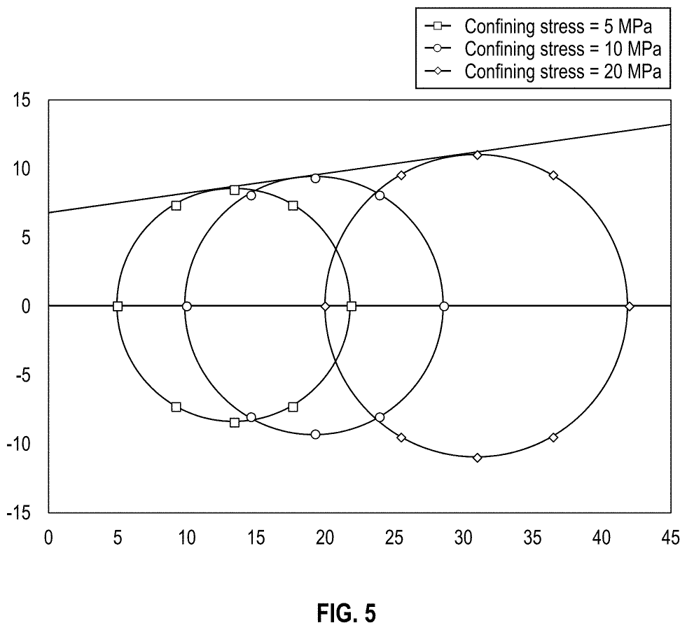

Simulated mechanical tests were performed on the digitized model 300 to determine mechanical properties and behaviors of the composite material. The simulated mechanical tests were performed on the digitized model 300 using FLAC3D™. For this example, simulated isotropic compression tests and simulated triaxial compression tests were performed on the digitized model 300. Details of the simulated tests are described in more detail later. FIGS. 4A and 4B are graphs showing results of a simulated isotropic compression test performed on the digitized model 300. The graphs shown in FIGS. 4A and 4B provide results for one constituent (Material 1) of the digitized model 300. In other words, FIGS. 4A and 4B show the mechanical response of a single voxel of the digitized model 300 corresponding to an individual constituent (Material 1). When all of the voxels are assembled to form the digitized model 300 (as shown in FIG. 3), the digitized model 300 behaves like a Mohr-Coulomb material (as shown in FIG. 5). This simulated isotropic compression test included several load-unload excursions. The load-unload excursions each included increasing pressure (applying load), allowing the model 300 to swell (unload), and then repeating the process. The graph shown in FIG. 4A provides the pressure vs. displacement curve across five load-unload excursions. The graph shown in FIG. 4B provides the specific volume vs. natural logarithm of pressure curve across five load-unload excursions. The mechanical behaviors of the other constituent materials were similar to that of Material 1.

FIG. 5 is a graph showing results of simulated triaxial compression tests performed on the digitized model 300. Three simulated triaxial compressions tests at confining stresses of 5 MPa, 10 MPa, and 20 MPa were performed on the digitized model 300. For each of the triaxial compression tests, the confining stress was applied to the sample (along the x-, y-, and z-axes), and the simulation test ran until the digitized model 300 reached equilibrium under isotropic compression conditions. The bottom boundary of the digitized model 300 was then changed to have a roller support condition (that is, exhibiting reaction forces normal to the supporting surface), and the top of the digitized model 300 was compressed by a constant downward velocity. The reaction force and normal stress at the top surface of the digitized model 300 were monitored over the course of the test. The simulation was stopped when the stress at the top surface leveled off, indicating that the peak compressive loading capacity of the model 300 under the given confining stress was reached.

At confining stress (σ₃, also referred as minimum principal stress) of 5 MPa and 20 MPa, the simulated compression tests demonstrated that the compressive strengths (σ₁, also referred as maximum principal stress) of the composite material were:

| | Test 1 | Test 2 |
|---|---|---|
| Confining Stress | σ₃⁽¹⁾ = 5 MPa | σ₃⁽²⁾ = 20 MPa |
| Compressive Strength | σ₁⁽¹⁾ = 21.9 MPa | σ₁⁽²⁾ = 42 MPa |

Assuming that the plastic mechanical behavior of the composite material conforms to the Mohr-Coulomb yielding criterion, the angle of internal friction can be calculated by Equation 2:

$$φ = {sin}^{- 1} ( \frac{\frac{{σ}_{1}^{( 2 )} - {σ}_{1}^{( 1 )}}{{σ}_{3}^{( 2 )} - {σ}_{3}^{( 2 )}} - 1}{\frac{{σ}_{1}^{( 2 )} - {σ}_{1}^{( 1 )}}{{σ}_{3}^{( 2 )} - {σ}_{3}^{( 1 )}} + 1} ) \qquad ( 2 )$$

The angle of internal friction ϕ calculated by Equation 2 using the results of Tests 1 and Tests 2, was 8.35° (0.146 radians).

The cohesive strength (c, also referred as cohesion) can be determined by finding the value of c that satisfies the Mohr-Coulomb yielding criterion shown in Equation 3:

$${σ}_{1} = \frac{1 + sin ( φ )}{1 - sin ( φ )} {σ}_{3} + 2 c \frac{cos ( φ )}{1 - sin ( φ )} \qquad ( 3 )$$

Using Equation 3, the cohesive strength c of the composite material was found to be 6.6 MPa. The cohesive strength c can also be determined by producing Mohr's circles on a shear stress vs. normal stress plot, based on the results of the triaxial compression tests. For the graph shown in FIG. 5, an additional test with a confining stress of 10 MPa was performed. The cohesive strength c can be determined by finding the y-intercept of the line that is tangent to all of the Mohr's circles (also referred as the Mohr-Coulomb failure envelope).

The unconfined compressive strength (UCS, also referred as uniaxial compressive strength) is the maximum axial compressive stress that a cylindrical sample of material (such as a composite material) can withstand under unconfined conditions (that is, a confining stress of 0). The unconfined compressive strength is equal to the second term of Equation 3, which is rewritten in Equation 4:

$$UCS = 2 c \frac{cos ( φ )}{1 - sin ( φ )} \qquad ( 4 )$$

The unconfined compressive strength calculated by Equation 4 was 15.2 MPa. The properties determined from the results of the triaxial compressive tests (such as the angle of internal friction ϕ, the cohesive strength c, and the unconfined compressive strength) can be expected to be equal to or approximately equal to the corresponding properties of the real rock sample because of the correlation between the digitized model 500 and the real rock sample.

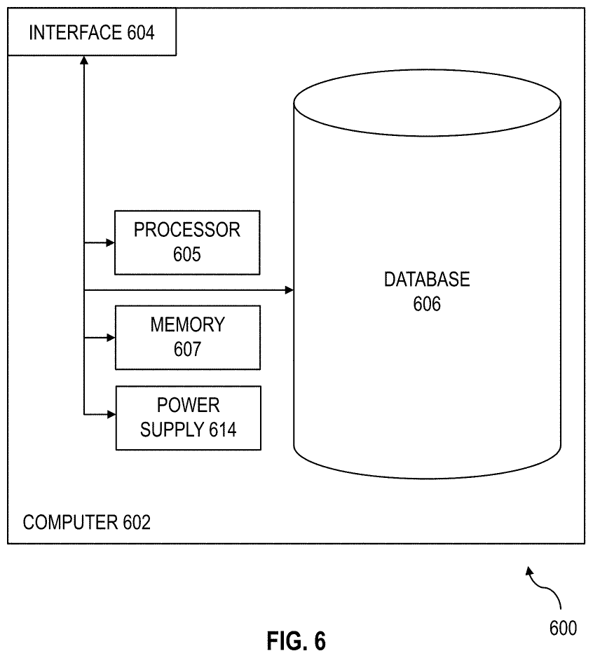

FIG. 6 is a block diagram of an example computer system 600 used to provide computational functionalities associated with described algorithms, methods, functions, processes, flows, and procedures, as described in this specification, according to an implementation. The illustrated computer 602 is intended to encompass any computing device such as a server, desktop computer, laptop/notebook computer, one or more processors within these devices, or any other suitable processing device, including physical or virtual instances (or both) of the computing device. Additionally, the computer 602 can include a computer that includes an input device, such as a keypad, keyboard, touch screen, or other device that can accept user information, and an output device that conveys information associated with the operation of the computer 602, including digital data, visual, audio information, or a combination of information. For example, the computer 602 can perform step 212 of method 200. In other words, the computer 602 can provide the mechanical properties determined at 210.

The computer 602 includes a processor 605. Although illustrated as a single processor 605 in FIG. 6, two or more processors may be used according to particular needs, desires, or particular implementations of the computer 602. Generally, the processor 605 executes instructions and manipulates data to perform the operations of the computer 602 and any algorithms, methods, functions, processes, flows, and procedures as described in this specification.

The computer 602 can also include a database 606 that can hold data for the computer 602 or other components (or a combination of both) that can be connected to the network. Although illustrated as a single database 606 in FIG. 6, two or more databases (of the same or combination of types) can be used according to particular needs, desires, or particular implementations of the computer 602 and the described functionality. While database 606 is illustrated as an integral component of the computer 602, in alternative implementations, database 606 can be external to the computer 602.

The computer 602 also includes a memory 607 that can hold data for the computer 602 or other components (or a combination of both) that can be connected to the network. Although illustrated as a single memory 607 in FIG. 6, two or more memories 607 (of the same or combination of types) can be used according to particular needs, desires, or particular implementations of the computer 602 and the described functionality. While memory 607 is illustrated as an integral component of the computer 602, in alternative implementations, memory 607 can be external to the computer 602.

The memory 607 stores computer-readable instructions executable by the processor 605 that, when executed, cause the processor 605 to perform operations, such as processing images (obtained, for example, by scanning a real composite material) to form a digitized model (such as the digitized model 300). The computer 602 can also include a power supply 614. The power supply 614 can include a rechargeable or non-rechargeable battery that can be configured to be either user- or non-user-replaceable. The power supply 614 can be hard-wired. There may be any number of computers 602 associated with, or external to, a computer system containing computer 602, each computer 602 communicating over the network. Further, the term “client,” “user,” “operator,” and other appropriate terminology may be used interchangeably, as appropriate, without departing from the scope of this specification. Moreover, this specification contemplates that many users may use one computer 602, or that one user may use multiple computers 602.

While this disclosure contains many specific implementation details, these should not be construed as limitations on the scope of the subject matter or on the scope of what may be claimed, but rather as descriptions of features that may be specific to particular implementations. Certain features that are described in this disclosure in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example implementations do not define or constrain this disclosure. Other changes, substitutions, and alterations are also possible without departing from the spirit and scope of this disclosure.

What is claimed is:

1. A method comprising:
for a real composite material comprising a plurality of real materials interspersed to form the real composite material:
determining, by one or more real mechanical tests, a plurality of mechanical properties of the respective plurality of real materials;
scanning the real composite material to form a digitized model of the real composite material, the digitized model of the real composite material comprising digitized models of the plurality of real materials;
assigning the determined plurality of mechanical properties to the corresponding plurality of digitized models of the respective plurality of real materials resulting in a modified digitized model of the real composite material;
performing one or more simulated mechanical tests on the modified digitized model of the real composite material;
determining a mechanical property of the real composite material based on results of the one or more simulated mechanical tests; and
providing the mechanical property of the real composite material.

2. The method of claim 1, wherein scanning the real composite material comprises scanning electron microscopy (SEM) imaging or micro computed tomography (micro-CT) imaging to create a plurality of images, and the digitized model of the real composite material is formed using the plurality of images.

3. The method of claim 2, wherein the digitized model of the real composite material is formed by identifying, in the plurality of images, digitized portions that correspond to each of the plurality of real materials.

4. The method of claim 3, wherein assigning the determined plurality of mechanical properties to the corresponding plurality of digitized models of the respective plurality of real materials comprises assigning the determined plurality of mechanical properties to the digitized portions identified as corresponding to each of the plurality of real materials.

5. The method of claim 4, wherein the real composite material comprises a core plug obtained from a subterranean zone.

6. The method of claim 5, wherein the plurality of real materials comprises organic material.

7. A method comprising:
for a composite material comprising a plurality of constituents interspersed to form the composite material:
forming a digitized model of the composite material, the digitized model comprising a plurality of digitized sub-models corresponding to the respective plurality of constituents;
assigning one or more mechanical properties to each of the digitized sub-models to form a modified digitized model of the composite material, the assigned mechanical properties each corresponding to mechanical properties of their respective constituents;
performing a simulated mechanical test on the modified digitized model to determine a mechanical property of the composite material; and
providing the mechanical property of the composite material.

8. The method of claim 7, wherein forming the digitized model of the composite material comprises:
scanning the composite material to obtain a plurality of images; and
compiling the plurality of images to form the digitized model of the composite material.

9. The method of claim 8, wherein scanning the real composite material comprises scanning electron microscopy (SEM) imaging or micro computed tomography (micro-CT) imaging.

10. The method of claim 9, wherein the composite material comprises a core plug obtained from a subterranean zone.

11. The method of claim 10, wherein the plurality of constituents comprises organic material.

12. The method of claim 10, further comprising performing one or more mechanical tests to determine one or more mechanical properties of each of the plurality of constituents, wherein the one or more mechanical properties determined by performing the one or more mechanical tests are the one or more mechanical properties assigned to each of the digitized sub-models.

13. A method comprising:
for a composite material comprising a plurality of constituents interspersed to form the composite material:
determining mechanical properties of each of the plurality of constituents;
scanning the composite material to obtain at least one image;
processing the at least one image to form a digitized model of the composite material, the digitized model comprising a plurality of digitized sub-models corresponding to the respective plurality of constituents;
modifying the digitized model to include the determined mechanical properties of each of the plurality of constituents, resulting in a modified digitized model;
determining a mechanical property of the composite material based on results of a simulated mechanical test on the modified digitized model; and
providing the mechanical property of the composite material.

14. The method of claim 13, wherein the composite material comprises a core plug obtained from a subterranean zone.

15. The method of claim 14, wherein the plurality of constituents comprises organic material.
